(19) United States
(12) Patent Application Publication
(10) Pub. No.: US 2020/0331824 A1
(43) Pub. Date: Oct. 22, 2020

(54) PROCESSES FOR CONVERTING C2-C5 HYDROCARBONS TO GASOLINE AND DIESEL FUEL BLENDSTOCKS

(71) Applicant: Swift Fuels, LLC, West Lafayette, IN (US)

(72) Inventors: Chris D'Acosta, West Lafayette, IN (US); Jeffery Miller, Naperville, IL (US); Robert Hoch, Hensonville, NY (US)

(73) Assignee: Swift Fuels, LLC, West Lafayette, IN (US)

(21) Appl. No.: 16/918,324

(22) Filed: Jul. 1, 2020

Related U.S. Application Data
(63) Continuation-in-part of application No. 16/738,290, filed on Jan. 9, 2020.
(60) Provisional application No. 62/790,175, filed on Jan. 9, 2019.

(51) Int. Cl. C07C 2/76; C10G 59/00; C07C 2/12; C10L 1/08; C10L 1/06

(52) U.S. Cl. CPC C07C 2/76; C10G 2300/1081; C07C 2/12; C10L 1/08; C10L 1/06; C10L 2200/0446; C10L 2270/026; C10G 2400/04; C10L 2200/0423; C10L 2270/023; C10G 2400/02; C10G 2300/4006; C10G 2300/4012; C10G 2300/4018; C10L 2290/06; C10L 2290/10; C10G 2300/4081; C10G 59/00

(57) ABSTRACT

Disclosed herein are processes for the production of hydrocarbon fuel products from C2-5 alkanes. Methane is converted to ethylene in a methane thermal olefination reactor operating at a temperature of at least 900° C. and a pressure of at least 150 psig, and without a dehydrogenation catalyst or steam. C2-5 alkanes are converted to olefins in a C2-5 thermal olefination reactor operating at a temperature, pressure and space velocity to convert at least 80% of the alkanes to C2-5 olefins. The ethylene and C2-5 olefins are passed through an oligomerization reactor containing a zeolite catalyst and operating at a temperature, pressure and space velocity to crack, oligomerize and cyclize the olefins. In one aspect, methane in the effluent of the oligomerization reactor is recycled through the C2-5 thermal olefination reactor. Methods for the thermal olefination of methane are also disclosed.

### CROSS-REFERENCE TO RELATED APPLICATION

This application claims the benefit of U.S. application Ser. No. 16/738,290, filed Jan. 9, 2020, which claims the benefit of U.S. Provisional Application No. 62/790,175, filed Jan. 9, 2019, both of which are hereby incorporated by reference.

### FIELD

Disclosed are processes for the conversion of C2-5 hydrocarbons to gasoline and diesel engine fuel blendstocks. C2-5 alkanes are converted to alkenes which are subsequently converted to produce targeted ranges of C₄ to C₁₆₊ fuel blendstocks. This invention offers a specialized thermal and multi-iterative chemical reaction process to transform any source comprising C₂+ light hydrocarbon streams into compounds for use as primarily gasoline blendstocks or diesel fuel blendstocks. The liquid effluent produced from the LG2F Processes can be specifically targeted by operating conditions and catalyst choices to yield any desired range of C₄ to C₁₂ gasoline compounds (i.e., high octane paraffins, olefins and aromatics) or as C₉ to C₁₆₊ high-performance middle distillate compounds (e.g. zero sulfur, high cetane, low pour point for use in ultra-low-sulfur diesel fuel).

### BACKGROUND

While the total demand for gasoline is steady or in a small level of decline, there is a rising demand for premium gasoline blendstocks to meet the needs of new more efficient higher-compression spark-ignited engines. There is also a rising demand of high-performance ultra-low sulfur diesel fuel blendstocks with high cetane values and effective cold-temperature flowability properties. These demands exist while surplus light hydrocarbons are readily available from midstream, refinery and petrochemical facilities for transformation to fuel grade products.

The petrochemical industry uses extremely complex, high-precision and capital-intensive methods to separate and purify chemical grade compounds. Consider that propane to propylene, or ethane to ethylene requires cryogenic separation (−100° C.) followed by ultrapure, dry, non-contaminated hydrogeneration processing to eliminate very-close boiling molecules (e.g., butadiene, propyne, acetylene) that can be highly reactive to their chemical processing and/or poison their polymerization catalysts. None of these items are a concern to the present process.

### SUMMARY

An aspect of this disclosure, referred to herein generally as the Light Hydrocarbon Gas to Fuel Process, or “LG2F”, converts any single or mixed stream of C₂₋₅ hydrocarbon compounds to C₄ to C₁₆₊ fuel grade hydrocarbons. The process uses a thermal olefination reaction followed by an acid-catalyzed oligomeric and cracking reaction. The process may be performed in a single or a multi-fixed-bed reactor.

This invention utilizes a thermal olefination reactor producing a series of dehydrogenation and cracking reactions to upgrade any source of light hydrocarbon gas phase compounds (i.e., C₂, C₃, C₄, C₅), to produce an olefin-rich light gas stream. Low-boiling olefin-rich gas compounds are then transformed to produce a spectrum of longer, branched-chain alkanes as well as aromatics, by using zeolite catalysts in a temperature controlled catalytic reactor. This transformation of light gases results in unique, higher-valued liquid streams including high-octane compounds for use as gasoline blendstocks or high-cetane compounds for use as diesel blendstocks.

The LG2F Process is extremely efficient and requires no complex distillation, cryogenics or hydrogenation processing.

The LG2F process eliminates the need for traditional complex multi-stage distillation, multi-stage cryogenic separation and selective hydrogenation (for chemical purification in the base petrochemical industry), while producing a diverse molecular spectrum of C₄ to C₁₆₊ blendstocks ideal for transportation fuels with up to 60% less capital investment.

The LG2F process converts off-gas compounds to liquid fuels and thereby minimizes the production losses attributed to low-value off-gas compounds. The sequencing of C2+ gas phase dehydrogenation processes can be tailored to maximize the yield of C4+ higher-octane blendstocks to meet regulatory fuel specification requirements.

### BRIEF DESCRIPTION OF THE DRAWINGS

FIG. 1 is a diagram showing the LG2F Process Flow for Alkane-Rich Light Gases.

FIG. 2 is a diagram showing the selectivity of thermal olefination of pentane.

FIG. 3 is a detailed diagram of the LG2F process flow.

FIG. 4 is a graph showing the carbon number distribution for Average Jet A.

FIG. 5 is a graph showing the typical carbon distribution for diesel fuel.

FIG. 6 is a diagram showing a process flow for a combination of the LG2F and I2FE processes.

FIG. 7 is a diagram showing the use of methane thermal olefination (MTO) with the LG2F Process.

FIG. 8 is a diagram showing an alternative LG2F process flow for alkene-rich light gases.

FIG. 9 is a graph showing chemical reaction selectivity of gasoline hydrocarbons by temperature.

FIG. 10 is a diagram showing the optional elimination of benzene from gasoline blendstocks.

FIG. 11 shows optional single and dual reactor systems for dewaxing.

FIG. 12 is a diagram showing a process flow for a low-severity method for a dewaxing process.

### DESCRIPTION

For the purpose of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Embodiments of the invention are shown in detail, but it will be apparent to those skilled in the relevant art that some features that are not relevant to the present invention may not be shown for the sake of clarity.

### OVERVIEW

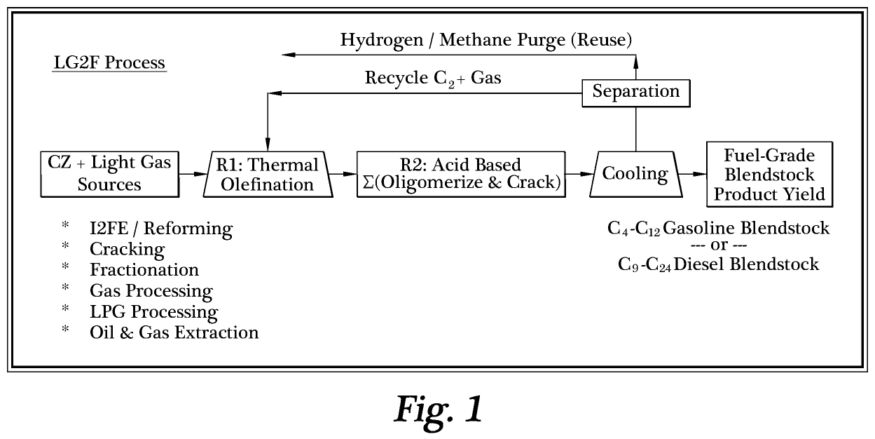

The LG2F Process is based upon a unique and efficient combination of a thermal olefination reaction to transform light paraffinic compounds to olefinic compounds without the requirement to use of a catalyst. The olefinic compounds are then catalytically transformed into the fuel-grade blendstock. The LG2F Process thus converts light hydrocarbon gases to high-grade transportation fuels that span a select range of hydrocarbon compounds and that possess certain fuel performance characteristics. See FIG. 1.

The overall LG2F Process is exemplified in FIG. 1. The C2+ Light Gas feed stream is directed to a thermal olefination chamber (R1) where it is combined with recycle light gases. Acid-based oligomerization, cracking, and aromatic cyclization take place in a second reactor (R2). Upon the completion of the C₂+ catalytic process the resulting hydrocarbon stream is condensed for liquid recovery. The hydrogen and methane in the cooled light gases from the cracking reactor are separated and purged for other uses. A recycle C2+ stream is combined with the C2+ feedstock and fed into the thermal olefination unit.

Fuel-grade hydrocarbons, preferably C6+ blendstock for gasoline and C9+ blendstock for diesel fuel is recovered. As a result, any C₁₊ light alkane streams of mixed gas or LPG type compounds can be transformed to any range of C₄ to C₁₆₊ hydrocarbon constituents for use in various transportation fuels, with methane and hydrogen as byproducts. Another feature of the light gas transformation is the creation of aromatic hydrocarbons which add energy density and bring a higher-octane value to the gasoline blendstock and contribute to thermal stability and seal swell in diesel fuel blendstock.

### Thermal Olefination

The LG2F processing steps are adaptable to convert different sources of light gases. The alkane gases are treated in a Thermal Olefination Reactor. The thermal olefination reactor is configured to dehydrogenate the alkanes to form olefins. The thermal olefination reactor favors the production of C₂+ olefins and other light hydrocarbon byproducts for a given feedstream. For example, pentane may be cracked into alkenes and paraffins as illustrated by the following examples:

C₅H₁₂→C₄H₈ (olefin)+CH₄ (paraffin)

C₅H₁₂→C₃H₆+C₂H₆

C₅H₁₂→C₂H₄+C₃H₈

C₅H₁₂→C₅H₁₀+H₂

Similarly, ethane may be cracked into an alkene with hydrogen as a byproduct, as illustrated by the following examples:

C₂H₆→C₂H₄ (olefin)+H₂

The result of the Thermal Olefination reaction, depending upon the composition of the light hydrocarbon feedstream, is a mix of C2 to C5 olefins and a smaller amount of C1 to C5 alkanes and hydrogen as byproducts. The conversion can be tailored to minimize the production of methane, as this byproduct is unproductive to the overall LG2F process.

### Olefination Operating Conditions

The olefination is performed at a high-temperature, with no catalyst required. The thermal olefination reactor is preferably operated at a temperature above 600° C., an internal pressure of 0-1500 psig, and a gas hourly space velocity of 30-1000. The C2+ thermal olefination process does not materially affect methane or hydrogen from the feed stream.

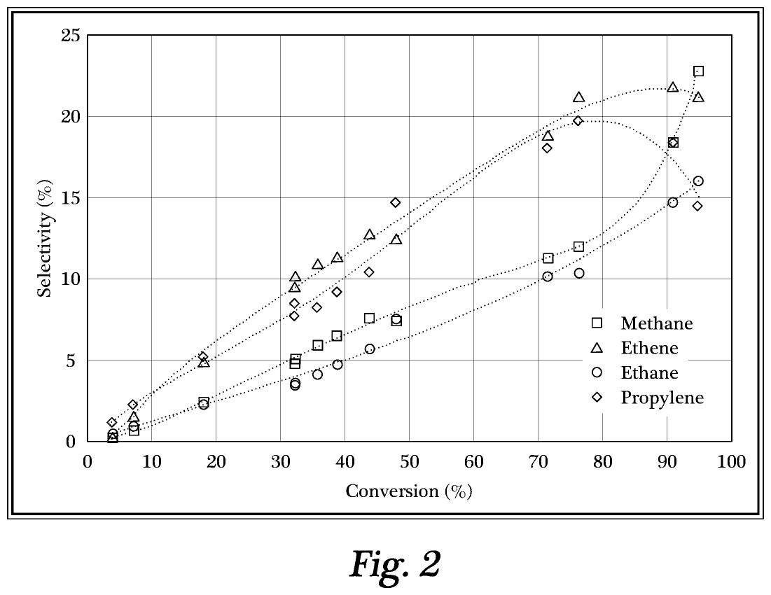

The results of an exemplary, single-pass LG2F processing of a C₅ alkane (pentane) feedstock is shown in FIG. 2. This demonstrates the ability to optimize operating conditions based on the composition of the feed stream. In particular, it shows the ability to target the production of higher-octane olefin compounds with some unreacted paraffin byproducts, while minimizing the production of methane. In one aspect, for example, this optimization is achieved at approximately 76% conversion, as shown in FIG. 2.

### C2-5 Alkane Feed Streams

The thermal olefination reactor receives and processes C2-5 alkane feed streams. As used herein, the term “C2-5” is used to refer to hydrocarbons having from 2 to 5 carbon atoms. The term “C2-5 alkane feed stream” or “C2-5 Stream” refers to a feed stream comprising alkanes having from 2 to 5 carbons, namely ethane, propane, butane and/or pentane. For example, a typical C2-5 Stream may include ethane, propane, n-butane, iso-butane and n-pentane.

Shorter chain alkanes are commonly referred to as “light (hydrocarbon) gases” since the C2-5 alkanes are gases at elevated temperatures (e.g. >36° C.). In a broad sense, the term “light gases” also encompasses alkenes. The term “liquid petroleum gas”, or LPG, is used to refer to light gases, primarily propane and/or butane, that may be contained in a liquid form. As used herein, the term “light gases” refers to a composition of any of the C2 to C5 alkanes and/or alkenes, although some of these compositions may be stored in a liquid form under pressurized conditions.

A C2-5 Stream may also include methane, although methane is not affected by the C2+ thermal olefination reaction. However, a separate provision may be made for use of methane in the LG2F Process, as detailed in the later described MTO Process.

A given C2-5 source may be processed as a standalone, or it may be combined with other available light gas streams for transformation to C₆₊ transportation fuel blendstocks. In one embodiment, the gas may be predominantly one single compound (e.g., ethane or propane, having been separated from an upstream operation).

The C2-5 Streams for use as feeds for the LG2F Process may additionally comprise any combination of other hydrocarbon types in addition to alkanes. For example, the C2-5 Stream may also include other hydrocarbons such as alkenes or aromatics. However, it will be appreciated that having a significant amount of components other than the C2 to C5 alkanes is not preferred, particularly if the other components will not be affected, or will be adversely affected, by the C2+ thermal olefination process. In an aspect, the C2-5 Stream contains alkanes having 2 to 5 carbons constituting at least 80 wt % of the C2-5 Stream, preferably at least 90 wt %, and most preferably at least 95 wt % of the C2-5 Stream.

In a different set of embodiments, the gas stream may be a mixture of any two or more alkane and alkene compounds where some type of pretreating may have occurred to eliminate unwanted compounds.

### C2-5 Sources

There are many diverse sources of C₂ to C₅ light hydrocarbon gas streams which may be obtained from natural gas liquids (NGLs), gas condensate, industrial fuel gas, petroleum gases and liquified petroleum gases (LPG), which are available across the oil, gas & petrochemical industry. Some streams are light gas compounds, typically containing ethane, propane, butane, pentane or any mixtures thereof. Pentane and butane/pentane mixtures may be in liquid form at ambient temperatures and pressures. Some sources are pre-treated to eliminate contaminates for downstream processing (i.e. H2O, H2S, N2, NH3). Some sources may be an isolated stream of virtually one compound (e.g. propane).

Suitable C2-5 sources are typically found in refineries, oil & gas extraction facilities, gas processing plants, petrochemical plants, and LPG storage facilities. C2-5 sources also include any light hydrocarbon gases output of catalytic cracking or catalytic reforming, or streams exiting any paraffin cracking unit. Any combination of suitable C2-5 light gas streams can be merged together to utilize this transformative LG2F reaction of liquids. Table 1 provides a more extensive list of potential C2-5 sources. These and other C2-5 sources are all eligible to be thermally and catalytically converted to C₆₊ constituents to maximize liquid volume yield of gasoline or diesel fuel blendstocks.

TABLE 1
Sample C2+ Gas Sources
Sources of C₂+ Hydrocarbon Gas Streams (Process Byproduct)

```
    Catalytic Reforming
    Paraffin Cracking
    Catalytic Cracking
    Gas Plants
    Gas Separation Units
    Hydrotreating Units
    Hydrodesulfurization Units
    Catalytic Isomerization Units
    Visbreaking Units
    Polymerization Units
```

### Reformate Source

In one example, the amount of long-chain cracked paraffin was between 3% to 14% (wt./wt.) hydrocarbon gases which are upgraded from low-value industrial fuel uses to become higher-value gasoline blendstocks by processing in the LG2F Process. Similar gas constituents (predominately C₂+ with hydrogen) are available from the output of the catalytic reformer, creating the opportunity for even larger liquid volume yields of high-octane gasoline blendstocks. These gas streams can be pretreated if necessary, and processed individually or merged with any number of other available gas streams.

### Pretreatment of Feed Streams Contaminates

In different embodiments, the feedstock may contain amounts of nitrogen, sulfur or any combination of these or other naturally occurring compounds. The feedstock may be unprocessed and may have inert compounds such as hydrogen or methane which are unreactive and are purged as byproducts in the LG2F Process. Some streams may contain contaminates which could poison certain catalysts or cause accelerated corrosion to downstream (refining or petrochemical) processing units. Larger concentrations of these contaminates may be removed in advance by pre-treatment.

As is often the case, contaminates such as sulfur, H₂S, nitrogen, water, etc. may exist with various field sources of the light hydrocarbon supply. Industrial sources, such as fuel gas supplies, may or may not be scrubbed to reduce such contaminates. Accordingly, the option exists in the configuration of LG2F Process to tailor the process to anticipate the quality and composition of the feedstream to optimize the processing configuration.

A pretreatment step is designed to eliminate any contaminates such as H₂S, NH₃ and H₂O found in the hydrocarbon source. However, this pre-treatment process may only be necessary if the catalyst options for C2-5 processing dictate that hydrocarbon pre-treatment is a necessary precursor to an effective LG2F operation. This pre-treatment process is not necessary when using clean light gas feedstocks, e.g., cracked gases from reformate, as these light hydrocarbon streams were treated upstream and contain ultra-low quantities of contaminates.

### C2-5Olefin Catalytic Processing

The C2+ thermal olefination step is followed by multi-iterative, acid-catalyzed, oligomerization, cyclization and cracking chemical reactions, which transform the olefin-rich compounds in the feedstream to a selected array of fuel grade hydrocarbons. The oligomerization, cracking, and aromatic cyclization processes productively convert the light olefins to a broad spectrum of fuel grade hydrocarbons without affecting the lighter (C_{2/}C₃) paraffins in the feed stream.

The broad-spectrum C2-5 olefin stream (and its byproducts) from the C2+ thermal olefination reaction is used as the feedstock to the catalytic processor. As used herein, the term “catalytic processor” or “catalytic reactor” is used to refer to a reactor using catalyst and operating under conditions to oligomerize, cyclize and crack the olefins to form higher carbon alkanes, alkenes and aromatics suitable for gas or diesel blending stocks. The catalytic reactor uses a zeolite catalyst operating above 200° C. at 0-1500 psig, and a weight hourly space velocity between 0.5 and 10 (preferably about 1). This reactor produces multi-iterative, random-sequenced chemical reactions to oligomerize, cyclize and crack the broad-spectrum of hydrocarbons. The catalytic process can be tailored to produce any range of fuel grade products, including for example C₅ + or C₆₊ or C₇₊ gasoline range (primarily paraffins, aromatics) or C₉₊ or C_{10 +} or C₁₂₊ range of light gas oil or middle distillate hydrocarbons for use primarily as diesel fuel blendstocks.

The chemical reactions in the catalytic R2 reactor comprise multi-iterative, unselective, building, cyclizing and degrading of different molecular formations creating a portfolio of hydrocarbons that can be selectively tailored to any specific carbon range of products using the recycle loop based upon the LG2F operating conditions. The operating conditions (T, P, WHSV) will vary depending upon the target product—gasoline grade or middle distillate grade fuel blendstocks.

The LG2F thermal olefination reaction (R1) along with the chemical reaction (R2) and recycle loop can be used independently, and can be interchangeably tailored based upon feedstock quality and targeted end products to produce gasoline blendstocks and/or diesel fuel blendstocks. The process is flexible to allow the reactor operating conditions to be established to produce the desired blend components and compositional features to meet fuel performance requirements (e.g. aromatics for gasoline octane value, cetane for diesel performance). The byproducts of the reaction may include methane and hydrogen.

This invention also allows the interchangeability to utilize olefin-rich light gases, e.g. FCC or petrochemical byproducts, as viable feedstreams directly into the R2 reaction which thereby produce a selected range of largely paraffinic and aromatic compounds and then return the C2-C5 light alkanes back via a recycle loop to process into the C2+ thermal olefination process. This sequence highlights that any olefin-rich light gases can utilize R2→R1→R2 to produce gasoline or diesel fuel blendstocks. Olefins typically have perceived high-value as petrochemicals building blocks for making polymers, however, this value can be nullified by location disparities, processing limitations and market fluctuations. Since most fuel grade products do not require the quality precision of ultra-fine chemicals, it may often be the case that unpurified fuel grade olefins are indeed preferred for use in fuel. The corollary to this is that C2-C5 light alkanes may be used to produce fuel grade olefins and aromatics via thermal olefination which are highly valued by petrochemical processors, and which bypass R2 for the production of fuel blendstocks.

### Catalysts

The catalytic processes disclosed herein utilize catalysts that oligomerize, cyclize and crack the olefins with high efficiency. The catalyst used in the LG2F Process generally contains a strongly acidic zeolite, with a high surface area support, for example, alumina. Additionally, there is a weakly active hydrogenation metal, for example molybdenum oxide, which saturates cracked olefins without saturation of aromatic compounds. By comparison, traditional catalytic naphtha reforming technology uses catalysts that contain platinum (Pt) on chloride alumina, often promoted with either tin (Sn) or rhenium (Re) for better yield and stability, respectively. These reforming catalysts are compositionally very different from the LG2F catalysts.

The LG2F Process uses catalysts which are functional to substantially oligomerize, cyclize and crack the olefins in the feed stream, while not significantly affecting other components of value in the feed stream. A catalyst is functional to substantially oligomerize, cyclize and crack the olefins if it transforms at least 80 wt % of the olefin light gases to fuel grade compounds, preferably at least 90 wt %, and more preferably at least 95 wt % of the olefin light gases.

In one embodiment, the catalytic process is performed uses a zeolite catalyst. The acidic cites in zeolite catalyze cracking reactions more rapidly than other components. The reactions can be conducted both with and without metal impregnation. The metal allows hydrogen to add across olefinic compounds.

In one aspect, the processes use a zeolite catalyst having a pore size of 2 to 8 Angstroms. Exemplary surface areas for the catalyst are 400 to 800 m2/gram. Examples of the zeolite catalysts include Si, Al and O, preferably with an Si:Al ratio of 10 to 300. Zeolite catalysts with properties outside of these limitations may also be useful. The catalyst is preferably selected to substantially catalyze the olefins while not significantly affecting other components of value in the feed stream.

In embodiments, the catalyst is Zeolite ZSM-5, Zeolite Beta or Zeolite Mordenite. Impregnations of these catalysts all use the same metal at varying concentrations for activity. Molybdenum trioxide is used to impregnate the zeolite catalyst with molybdenum. This creates a bifunctional catalyst that is an acid and metal. Zeolites are characterized in the following ways: pore size—3 to 8 angstroms usually; pore structure—many types; and chemical structure—combination of Si, Al, and O. All have ammonium cations (except one version of mordenite) until impregnation and all have molar Si/Al ratios of 10 to 300.

Zeolite Beta has the following properties: 2-7 angstroms pore size, SiO2 to Al2O3 molar ratio (Si/Al) ranging from 20 to 50, intergrowth of polymorph A and B structures, and surface area between 600 and 800 m2/gram.

Zeolite Mordenite has the following properties: 2-8 angstroms pore size, sodium and ammonium nominal cation forms, Si/Al ratio of 10 to 30, and surface area between 400 and 600 m2/gram.

In a particular embodiment, the catalyst is Zeolite ZSM-5. ZSM-5 has the following properties: 4-6 angstroms pore size, pentasil geometry forming a 10-ring-hole configuration, Si/Al ratio of 20 to 280, and surface area between 400 and 500 m2/gram. Various impregnations may use between 1% and 2% molybdenum. Our research found the ZSM-5 was the preferred catalyst for its ability to support the R2 transformation reaction while preserving the chemical composition of the aromatic compounds. The reaction can be conducted both with and without metal impregnation. The metal allows hydrogen to add across olefinic compounds that are produced during the cracking mechanism. The smaller pore size of the ZSM-5 catalyst results in far undesired saturation of aromatic compounds, which are a generally desired constituents in both gasoline and diesel blendstocks.

### Zeolite Catalyst Example

The proprietary acid-based (ZSM-5 zeolite) catalyst specifically targets C₂-rich hydrocarbon streams (e.g. one embodiment: 80% silica on alumina). The process design may also have catalyst beds which favor C₂ reactions more than C₃ reactions or C₄ reactions resulting in layers or sequences of oligomerization and cracking reactions with different conditions to maximize the yield and performance properties of the fuel products.

### Recycle

Following the R2 catalytic reaction, the alkane-rich light gas recycle stream exiting the flash drum condensation unit can be directed back to the C₂+ thermal olefination reactor to be merged with other incoming light hydrocarbon streams as depicted in the process flow (FIG. 1). The constituents outside the selected array are gathered into a single-loop recycling configuration. This recycle process minimizes the cost and maximizes the yield profile and performance properties of any type of the liquid effluent produced for transportation fuel use. Typically, for all compounds not used in a targeted gasoline range (e.g. C₆₊) or diesel fuel range (e.g. C₁₀₊), the process will direct the lighter byproducts (e.g. ≤C₆ or ≤C₁₀) to be recycled for further upgrading.

This recycle loop is multi-iterative as necessary to allow the random redistribution of C₆₊ liquid hydrocarbons yielded from LG2F to unite in various formations (e.g., paraffins, olefins, aromatics) needed for a fuel based upon specific performance characteristics. Such performance characteristics for gasoline might include octane, vapor pressure, density, net heat of combustion, etc., while such characteristics for diesel fuel might include cetane, thermal stability, cold flowability, and others.

An optional feature of LG2F is to produce C₄ and C₅ alkanes which may be useful for increasing the volatility and raising the vapor pressure in gasoline, although often at the expense of octane levels. Thus, some or all of the C4-5 alkanes may be targeted for production into the gasoline blendstock. Alternatively, C4 or C4-C5 production may be avoided, in which case the process directs C₄ or C₅ byproducts to be recycled for further upgrading.

### LG2F Products

The process configuration utilizes a recycle loop to produce a specified range, for example C₆ to C₁₂ gasoline compounds or C₁₀ to C₂₀ diesel fuel compounds for use as blendstocks in high grade transportation fuels. Using the LG2F process, the liquid volume yields can range from 65% to 95+ wt % of the initial gas stream (wt %) depending upon the severity of operating conditions. This process offers flexibility in making paraffinic molecules of higher yield, or olefinic molecules and aromatic hydrocarbons of somewhat lower yields for gasoline range products, or alternatively, it can be switched to create a blend of middle distillates (primarily paraffins and aromatics) primarily for diesel range products.

### By-Products

In all LG2F embodiments, excess methane and hydrogen are byproducts of the thermal olefination reaction. Since methane and hydrogen are unreactive to the LG2F process, there is no restriction on their being present in the light hydrocarbon gas feedstream.

The LG2F Process will produce varying amounts of methane, which is generally lower-value and therefore undesirable. Depending upon the C₂₊ feedstock quality, the LG2F process provides the option of extracting excess methane and hydrogen via membrane separation. Produced H₂ is desirable if reusable as a byproduct.

### LG2F System

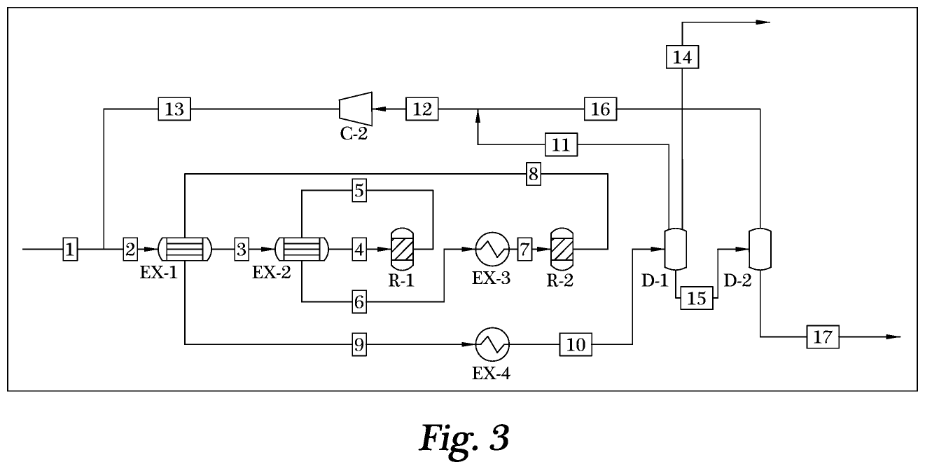

Referring to FIG. 3, there is shown a process flow for the LG2F Process. Feedstock stream (1) comprises mostly C₂-C₅ paraffin-rich hydrocarbons. Pretreatment (not shown) of the feed (1) can be conducted to remove contaminates for gasoline production or to optimize feed composition. Feedstock stream (1) is combined with a recycled light stream (13) comprised of a C₁-C₅ mixture primarily including n-paraffins and i-paraffins with some olefins and the combined stream (2) is fed into heat exchanger (EX-1). Light gas feed streams that have primarily olefin-rich content (e.g., FCC off-gases, propylene, etc.) can be fed directly into R-2 via line (7), bypassing the thermal olefination step, and then resume the recycle process via (8). The combined stream (2) is cross exchanged in EX-1 with stream (8), to recover heat produced in the catalytic reactor R-2. The outlet (3) of EX-1 is fed into another cross exchanger, EX-2, to further pre-heat the feed for R-1.

The pre-heated stream (4) is fed into a thermal olefination furnace (R-1) typically operating at 600-1100° C. and 0-1500 psig. Thermal olefination reactor (R-1) conducts an endothermic reaction to produce olefinic compounds via carbon cracking and dehydrogenation. Excess heat from the reaction is used as the hot stream (5) for EX-2. The hot stream (6) exiting EX-2 may require additional cooling for the second reaction stage (R-2). EX-3 is an optional air-water or refrigerant-based cooling unit for the system depending upon heating requirements. It is useful to here conduct the appropriate heat transfer step to ensure proper set-point R-2 inlet conditions. A bypass can be implemented between streams (6) and (7) and streams (9) and (10) in lieu of cooling utility for EX-3 and EX-4 for dynamic operability between diesel and gasoline production. An optional knockout step may be incorporated prior to the R-2 reactor in stream (7) to capture entrained liquid droplets and remove all C6+ compounds from entering R-2.

R-2 is catalytic reactor, typically operating at 200-1000° C. and 0-1500 psig, that oligomerizes, cyclizes and cracks olefinic compounds in multi-iterative reactions to produce a broad spectrum of n-paraffins, i-paraffins, naphthenes, and aromatics primarily across the C₄ to C₁₆₊ range, resulting in high-octane gasoline or high-cetane diesel spectrum products. Depending upon the final product desired, excess C₂ to C₁₂ compounds from this catalytic reaction can be recycled into fuel grade constituents. The reaction is very exothermic and can be configured with or without inter-stage cooling to prevent overheating. The excess heat from the reacted stream (8) is used in EX-1 as the hot stream inlet to step up temperature for the combined feed (2).

The hot outlet (9) can support optional cooling for proper flashing in flash drum D-1. For this reason, EX-4 may not be required but it could be an air-cooler, water cooler, etc. to conduct appropriate heat exchange. The flash drum feed (10) is kept at the pressure of the system and is used to purge targeted light components from the mixed product stream. The primary function of D-1 is to control the pressure of the system. Light components (11, 14) consist of mostly H2 and C1-C3 compounds that can either be purged (14) from the system or directly recycled (11) back into the system by combining with the flash drum (D-2) lights stream (16) prior to compressor, C-1.

D-1 light streams will have H2 and C1 components which are unreactive for the system and will cause accumulation in the recycle if not properly removed. H2 and C1 can be purged (14) with other light components to stabilize the recycle system or a separator, such as a membrane, can be utilized to selectively remove H2 and C1. The liquid bottoms (15) from D-1 are fed into D-2 which is set at a lower pressure to remove mostly C3 and C4 compounds from the liquid stream (15). Lights (16) from D-2 are combined with lights (11) from D-1 to form stream (12) which is compressed in C-1 and recycled for further reaction. Recyclable light hydrocarbons (16) from D-2 (typically C₂-C₄ if targeting gasoline; C₂-C₁₀ if targeting diesel) will be fed back to the thermal reaction, unless the constituents are olefin-rich which can optionally be fed directly into R-2 to increase process efficiency. The resulting flashed liquid stream (17) exiting the bottoms of D-2 is the final product of the process which can be targeted to produce any range of C₄-C₁₂ high-octane gasoline blendstock or C₉-C₁₆₊ high-cetane diesel fuel blendstock.

By way of example, the fully-recycled thermal and chemical reactions from processing C5 (pentane) are depicted in a material balance as shown below in Table 2a. The resulting C₆₊ gasoline compounds yielded a 52% mass conversion (14/52% mass as aromatics) from the C₅ feed and resulted in an unexpectedly high 101.7 Research Octane number (using ASTM D2699 Test Method). This illustration using C₅ as the feed to thermal olefination demonstrates the broad range of gasoline blend compositions that are possible. A similar example shown in Table 2b depicts C2 (ethane) with a 73% mass conversion to C5+ gasoline (17/73% mass as aromatics). This demonstrates the broad spectrum of molecular outcomes typical of all C2-5 feed streams. The C₂ to C₅ feedstocks can be fully recycled and converted to gasoline range molecules based upon the unique operating conditions of the reactor.

This illustration also depicts how specific operating conditions can be used to control the resulting slate of compounds. The temperature of Reactor 2 was 250° C. which resulted in a 40% m/m aromatic content. The aromatic content is variable and can be used to increase octane values of gasoline blendstocks. Surplus C6+ aromatics can be captured from the knockout as byproducts for petrochemical processing. Increasing the temperature of reactor 2 from 250° C. to 400° C. doubles the content of desirable aromatics in the gasoline blendstock and thereby increases the resulting octane. The lights purge (via flash drum and membrane separation) allows methane and hydrogen byproducts to be reused in other downstream processes.

TABLE 2a
Production of Gasoline Blendstock from C5 (pentane) feedstock:

| | Process Step | | | | | | | | |
|---|---|---|---|---|---|---|---|---|---|
| | 1 | 2 | 3 | 4 | 5 | 6 | 7 | 8 | 9 |
| LG2F using C5 | | R1 | | R2 | R2 | Flash | | Lights | |
| w/Recycle (lb/hr) | Feed | Out | Knockout | Feed | Out | Tops | Recycle | Purge | Gasoline |
| H2 | | 0.38 | | 0.38 | 0.38 | 0.38 | | 0.38 | |
| C1 | | 35.76 | | 35.76 | 35.76 | 35.76 | | 35.76 | |
| C2 | | 94.17 | | 94.17 | 30.99 | 30.99 | 30.99 | | |
| C3 | | 58.71 | | 58.71 | 19.29 | 19.29 | 19.29 | | |
| C4 | | 17.67 | | 17.67 | 42.42 | 42.42 | 42.42 | | |
| C5 | 100 | 79.36 | | 79.36 | 104.98 | 104.98 | 104.98 | | |
| C6 | | | | | 25.62 | | | | 25.62 |
| C7 | | | | | 7.24 | | | | 7.24 |
| C8 | | | | | 4.1 | | | | 4.1 |
| C9 | | | | | 0.33 | | | | 0.33 |
| C10 | | | | | | | | | |
| C11 | | | | | | | | | |
| C12 | | | | | | | | | |
| A6 | | 7.24 | 7.24 | | 0.99 | | | | 0.99 |
| A7 | | 2.74 | 2.74 | | 4.55 | | | | 4.55 |
| A8 | | | | | 6.56 | | | | 6.56 |
| A9 | | | | | 2.51 | | | | 2.51 |
| A10 | | | | | | | | | |
| A11 | | | | | | | | | |
| Unknown | | 1.67 | 1.67 | | | | | | |
| Total | 100 | 298 | 12 | 286 | 286 | 234 | 198 | 36 | 52 |

TABLE 2b
Production of Gasoline Blendstock from C2 (ethane) feedstock:

| | Process Step | | | | | | | | |
|---|---|---|---|---|---|---|---|---|---|
| | 1 | 2 | 3 | 4 | 5 | 6 | 7 | 8 | 9 |
| LG2F C2 with | | R1 | | R2 | R2 | Flash | | Lights | |
| Recycle (lb/hr) | Feed | Out | Knockout | Feed | Out | Tops | Recycle | Purge | Gasoline |
| H2 | | 2.94 | | 2.94 | 2.94 | 2.94 | | 2.94 | |
| C1 | | 26.42 | | 26.42 | 26.60 | 26.60 | | 26.60 | |
| C2 | 100 | 210.47 | | 210.47 | 211.20 | 211.20 | 211.20 | | |
| C2= | | 157.06 | | 157.06 | 25.51 | 25.51 | 25.51 | | |
| C3 | | 0.44 | | 0.44 | 0.44 | 0.44 | 0.44 | | |
| C3= | | 3.27 | | 3.27 | | | | | |
| C4 | | 0.75 | | 0.75 | 47.45 | 47.45 | 47.45 | | |
| C4= | | 0.48 | | 0.48 | 2.65 | 2.65 | 2.65 | | |
| C5 | | | | | 33.80 | 16.90 | 16.90 | | 16.90 |
| C6 | | | | | 15.84 | | | | 15.84 |
| C7 | | | | | 9.53 | | | | 9.53 |
| C8 | | | | | 5.39 | | | | 5.39 |
| C9 | | | | | 0.44 | | | | 0.44 |
| C10 | | | | | | | | | |
| C11 | | | | | | | | | |
| C12 | | | | | | | | | |
| A6 | | 0.67 | 0.67 | | 1.30 | | | | 1.30 |
| A7 | | | | | 6.82 | | | | 6.82 |
| A8 | | | | | 8.63 | | | | 8.63 |
| A9 | | | | | 3.31 | | | | 3.31 |
| A10 | | | | | | | | | |
| A11 | | | | | | | | | |
| Unknown | | 1.64 | 1.64 | | | | | | |
| Total | 100 | 404.16 | 2.32 | 401.84 | 401.84 | 333.70 | 304.16 | 29.54 | 68.15 |

Note:
“=” designates alkenes

### Gasoline Blendstocks

In one aspect the LG2F Process is tailored to the production of gasoline blendstocks, as exemplified in the foregoing discussion. As used herein, the term “gasoline blendstock” refers to a formulation comprising n-paraffins, iso-paraffins, cyclo-paraffins, olefins and aromatics having 4 to 12 carbons. See Table 3. The gasoline blendstocks from this invention preferably have 5-12 carbons, and more preferably comprise 6-11 or 7-10 carbons. The gasoline blendstocks also typically have branched-chain paraffins and aromatic hydrocarbons having 6 to 11 carbons, preferably 7 to 10 carbons. In preferred embodiments, the LG2F Process yields a product containing at least about 65 wt % C5-10 branched-chain paraffins and at least 25 wt % C7-9 aromatic hydrocarbon compounds. The following examples further demonstrate the ability to tailor the LG2F Process depending on the C2-5 feed stream and the desired end product(s).

TABLE 3
Typical Gasoline Composition

| Typical Gasoline | # of Carbon Atoms | | | | | | | | |
|---|---|---|---|---|---|---|---|---|---|
| Constituents | C4 | C5 | C6 | C7 | C8 | C9 | C10 | C11 | C12 |
| n-paraffins | XX | XX | XX | X | X | X | X | X | X |
| iso-paraffins | XX | XX | XX | XX | XX | XX | XX | X | X |
| cyclo-paraffins | | XX | XX | XX | XX | XX | XX | X | X |
| olefins | | XX | XX | XX | XX | XX | XX | X | X |
| aromatics | | | X | XX | XX | XX | XX | X | X |

### 2-5 Hydrocarbons to C6-8 Aromatics

In an embodiment, the LG2F Process is tailored by isolating the catalytic R2 reaction to convert C₂-C₅ light olefin feedstocks into aromatic hydrocarbons in a narrow range of solely C₆ to C₈ aromatics. This is done by use of operating conditions to obtain an aromatic yield up to the upper boiling limit of o-xylene, for example 145° C., and recycling all byproducts in the flash drum with boiling points below benzene at 80° C. The yield of C₆ to C₈ aromatics is valuable to the petrochemical market as a base aromatic feed stream to aromatics fractionation or as an alternative, if the BTX product stream is first processed by a hydrodealkylation step to decouple and remove ethyl-propyl and butyl-aromatic constituents leaving only methyl-aromatic products.

### C2-5 Hydrocarbons to C7-8 Aromatics

In another embodiment, this invention can be tailored by isolating the catalytic R2 reaction to convert C₂-C₅ light olefin feedstocks into aromatic hydrocarbons in a narrow range of C₇ to C₈ aromatics. Again, this is done by targeting the aromatic yield up to the upper boiling limit of o-xylene, for example 145° C., and recycling all byproducts in the flash drum with boiling points below toluene at 110° C. The yield of C₇ and C₈ aromatics have a very high-octane value and a very high energy density in the absence of benzene and are useful gasoline blendstocks to meet premium high-octane grades.

### C2-5 Hydrocarbons to C8 Aromatics

In another embodiment, the LG2F Process is tailored by isolating the catalytic R2 reaction to convert C₂-C₅ light olefin feedstocks into aromatic hydrocarbons in a narrow range of solely C₈ aromatics by targeting operating conditions for the aromatic yield up to the upper boiling limit of o-xylene, for example 145° C., and recycling all byproducts in the flash drum with boiling points below p-xylene at 138° C. The yield of C₈ aromatics will have a very high-octane value and a very high energy density which can be a useful gasoline blendstock to meet premium high-octane grades. In addition, these C₈ compounds may be further valuable to the petrochemical market, particularly if they are produced by a hydrodealkylation step to decouple and remove any close-boiling ethyl-aromatic constituents and produce methyl-aromatic products.

### C2-5 Hydrocarbons to C7-9 Aromatics

In one embodiment, this invention is tailored by isolating the catalytic R2 reaction to convert C₂-C₅ light olefin feedstocks into aromatic hydrocarbons in the C₇ to C₉ range by specifying operating conditions for the aromatic yield up to the upper boiling limit of trimethylbenzenes, for example 175° C., and recycling all byproducts in the flash drum with boiling points below toluene at 110° C. The yield of C₇ to C₉ aromatics will have a very high-octane value and a very high energy density, without the presence of benzene, and can be a useful gasoline blendstock to meet premium high-octane grades.

### C2-5 Hydrocarbons to Isooctane

One specialized technique to produce high-octane gasoline blendstocks is the use of LG2F in a truncated fashion—by setting the operating conditions of the catalytic R2 chemical reaction to the targeted upper temperature on the desired product stream. All light hydrocarbon gases below a lower targeted boiling point limit are recycled, creating a desired range of product. This technique allows production of a simple narrow band of desirable hydrocarbons that may be particularly valuable to the fuel blending process of a particular LG2F production facility.

One such example of the optionality is the targeting of isobutane, a high-octane compound typically used to add vapor pressure (RVP) to gasoline blending, but also used as a feedstock to any traditional paraffin alkylation process. The catalytic R2 chemical reaction favors the production of branched-chain paraffins, which reduces the likelihood of producing n-paraffins which boil on either side of isobutane. Accordingly, as a result of the tailored LG2F R2 reaction, isobutane (H₄H₁₀) can be isolated within a boiling range of between −40° C. and −2° C. All the lights below −40° C. (notably ethane and propane) are recycled to maximize the yield of branched paraffins within the temperature band.

In a similar example, the LG2F R1 thermal olefination reactor in this invention can be targeted to produce any combination of C₃-C₅ olefins (propene, butene and/or amylene) from any C3-C5 light gas alkanes which can then be directly applied into any traditional paraffin alkylation unit with the additional feed of isobutane (from any source) for production of high-octane, branched-chain paraffinic hydrocarbons, particularly 2,2,4-trimethylpentane (Isooctane).

In a combined example, the LG2F R1 thermal olefination reaction can be processed using any C3-C5 alkane gases to produce C3-C5 olefins. The ≤C3 stream can be extracted and processed by R2 (with the option to add additional light olefin streams) to target the production of isobutane as described above. Any C6+ byproducts from the R1 reaction can be captured by liquid-vapor knockout for surplus or reuse as petrochemical feedstock. This tailored configuration results in the critical feedstreams necessary for input to paraffin alkylation. Accordingly, this LG2F invention coupled with any paraffin alkylation reaction converts any C3-C5 light alkane gases to isooctane with methane, hydrogen and surplus aromatics as byproducts.

Paraffin alkylation units typically process an isoparaffin (e.g. isobutane) and low-molecular-weight alkenes in the presence of a strong liquid acid catalyst (typically H₂SO₄ or HF). Heterogeneous catalysts such as zeolites can also be utilized. In this example, the alkylation unit catalyst transfers the protons of the alkenes to produce carbocations which alkylate isobutane. The product of the alkylation reaction is typically 2,2,4-trimethylpentane, an ideal high-octane, aliphatic gasoline blendstock. Light alkane byproducts of the alkylation process are further candidates for use in LG2F R1+R2 processing for conversion to gasoline or diesel fuels as outlined in this invention.

### Diesel Blendstocks

### Background

Diesel fuel has several key performance characteristics which depend upon the chemical composition of the fuel. Diesel fuels are generally comprised of n-paraffins, iso-paraffins, cycloparaffins and aromatics in such a way as to meet key performance requirements of the fuel. For example, in a diesel engine, cetane number is the measure of the speed of the compression ignition upon injection of the fuel, as well as the quality of the fuel burn in the combustion chamber. Accordingly, a high-performance diesel fuel is preferred to have an aggregate cetane index value (using ASTM D613) of at least 40 and as high as 60.

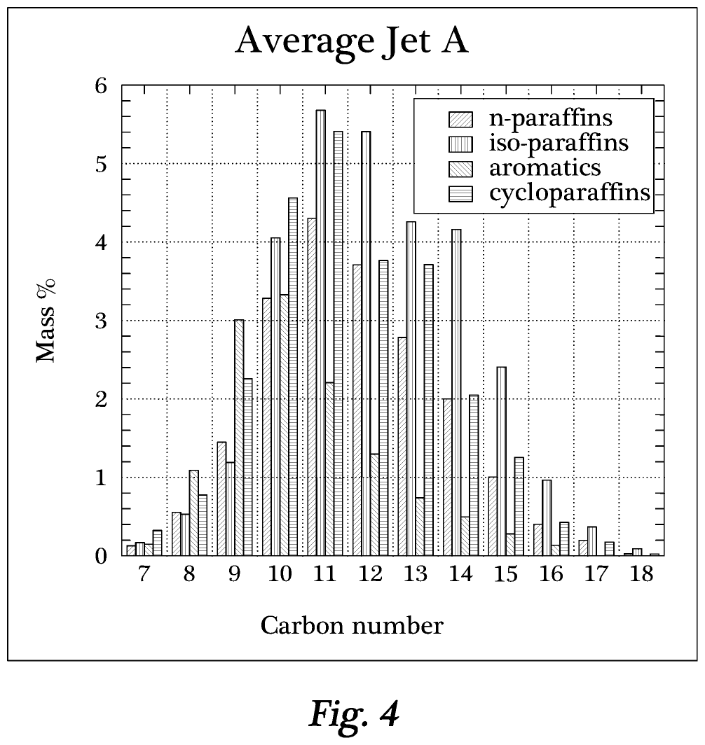

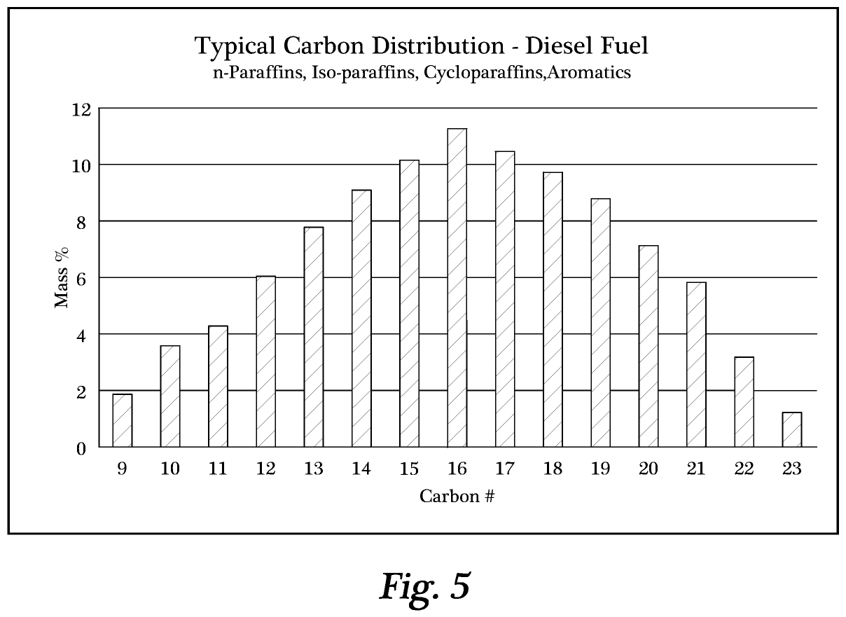

In addition, very low sulfur levels are also highly desirable in diesel fuel to eliminate corrosive wear-and-tear and prevent engine emission control system issues. Jet fuel and diesel fuel, both derived from middle distillates, share many common features. However, ASTM International fuel specifications call for different performance-based fuel test results impacting cetane, lubricity, viscosity, low temperature flowability, sulfur content, heating value, and more. The performance requirements are what dictate the composition and operating requirements to produce the desired fuel. See FIG. 4 showing the carbon number distribution for Average Jet A, and FIG. 5 showing typical carbon distribution for diesel fuel.

### Paraffins

Generally, C₉₊ n-paraffins, iso-paraffins and cycloparaffins have higher cetane values than aromatics and are key constituents in the diesel blendstock to achieving high cetane measures (e.g. 40-60) for good fuel performance. Cetane Values for various n-paraffins are shown below in Table 4.

TABLE 4
C9+ n-Paraffin Compounds Have Highest Cetane Values

| C9 to C20 | | Boiling Pt | Melting | |
|---|---|---|---|---|
| n-Paraffins | Formula | (° C.) | Point (° C.) | Cetane # |
| N-NONANE | C9H20 | 150 | −48 | 72 |
| N-DECANE | C10H22 | 174 | −30 | 76 |
| N-UNDECANE | C11H24 | 196 | −26 | 81 |
| N-DODECANE | C12H26 | 216 | −10 | 87 |
| N-TRIDECANE | C13H28 | 235 | −5 | 90 |
| N-TETRADECANE | C14H30 | 254 | 6 | 95 |
| N-PENTADECANE | C15H32 | 271 | 10 | 96 |
| N-HEXADECANE | C16H34 | 287 | 18 | 100 |
| N-HEPTADECANE | C17H36 | 302 | 22 | 105 |
| N-OCTADECANE | C18H38 | 316 | 28 | 106 |
| N-NONADECANE | C19H40 | 336 | 32 | 110 |
| N-EICOSANE | C20H42 | 344 | 36 | 110 |

However, while C14+ n-paraffins have high Cetane Values, their melting point is above low ambient temperatures. Specialized pour point, cloud point and cold filter plugging tests often call for a reduction of heavier n-paraffinic compounds in middle distillates (often via dewaxing) to improve the cold flowability and operability of a diesel fuel. In addition, n-paraffins have lower volumetric heating value (btu/gal) in comparison to aromatics.

Unlike gasoline for spark-ignited piston engines, which depend upon C₇-C₉ high-octane aromatics to retard early ignition, C₁₀ to C₂₀ aromatics provide diesel engines thermal stability, heating value (btu/gallon) and desirable elastomer swell characteristics. Unfortunately, these aromatics generally have low cetane values which can impede effective diesel engine performance. See Table 5.

TABLE 5
C10+ Aromatic Compounds Cetane Values

| | | Boiling Pt | Melting | Cetane |
|---|---|---|---|---|
| C10 to C20 Aromatics | Formula | (+ C.) | Point (° C.) | # |
| N-BUTYLBENZENE | C10H8 | 183 | −88 | 6 |
| 1-METHYLNAPHTHALENE | C11H10 | 245 | −30 | 0 |
| N-PENTYLBENZENE | C11H16 | 205 | −75 | 8 |
| N-HEXYLBENZENE | C12H18 | 226 | −61 | 19 |
| N-HEPTYLBENZENE | C13H20 | 246 | −48 | 35 |
| 1-N-BUTYLNAPHTHALENE | C14H16 | 289 | −20 | 6 |
| N-OCTYLBENZENE | C14H22 | 264 | −36 | 32 |
| N-NONYLBENZENE | C15H24 | 282 | −24 | 50 |
| N-DECYLBENZENE | C16H26 | 298 | −14 | |
| N-UNDECYLBENZENE | C17H28 | 313 | −5 | |
| 2-N-OCTYLNAPHTHALENE | C18H24 | 352 | −2 | 18 |
| N-DODECYLBENZENE | C18H30 | 328 | 3 | 68 |
| N-TRIDECYLBENZENE | C19H32 | 341 | 10 | |
| N-TETRADECLYBENZENE | C20H34 | 354 | 16 | 72 |

It is therefore desirable to be able to produce diesel blendstocks that primarily contain high Cetane Value components (e.g. C₉-C₁₆₊ n-paraffins) with lesser targeted amounts of aromatics (e.g. C₉-C₁₆) whose lower melting points help increase cold flowability of the fuel.

These varying factors and fuel requirements call for flexibility in the compositions of diesel fuels. In an aspect, the LG2F Process is tailored to the production of diesel blendstocks. As used herein, the term “diesel blendstock” refers to a formulation comprising n-paraffins, iso-paraffins, cyclo-paraffins and aromatics having 9 to 24 carbons. The diesel blendstocks preferably have 10-20 carbons preferably have less than 35 wt % aromatic hydrocarbons, and more preferably less than 30 wt %. The following discussion further demonstrates the ability to tailor the LG2F Process depending on the C2-5 feed stream and the desired diesel product(s).

This invention can be tailored by isolating the LG2F R2 chemical reaction to convert C₂-C₅ light olefin feedstocks into any range of C₉ to C₂₄₊ middle distillate hydrocarbons, ideally for diesel fuel blending. When using olefin feedstocks from any source with the LG2F R2 reactor for producing diesel fuel blendstocks, the acid-based chemical reaction produces a broad-spectrum of paraffin, iso-paraffin, cycloparaffin and aromatic output in a normal (gaussian) distribution. The distribution of the final product can be wide (e.g. C₉ to C₂₄₊) or narrowed (e.g. C₁₀ to C₁₇) depending upon the desired performance characteristics of the middle distillate blendstock.

For example, one embodiment targets the LG2F R2 product yield by setting the operating conditions to produce hydrocarbons up to the upper boiling limit of n-hexadecane for example 295° C. and recycling all byproducts in the flash drum with boiling points just above C₉ n-nonane at for example 145° C. This will yield a very high cetane blendstock with limited need for dewaxing. This can be a very useful premium diesel fuel blendstock, particularly if processed in the absence of any sulfur contaminates (e.g. using the optional C₂-C₅ light gas feeds from the I2FE long-chain cracking process). The lower carbon paraffins have low freezing points which improve fuel flowability in cold weather (pour point). Many other LG2F R2 operating conditions may also be utilized to optimize the fuel performance characteristics (e.g. cetane, pour point, density, heat of combustion, thermal stability, etc.) of the R2 product as a blendstock in comparison with other possible middle distillate blending components. The LG2F R1 and R2 reactions can be used together in a recycle loop or independently depending upon the availability of the alkane or alkene light gas feedstreams. Assessing the middle distillate product requirements in relation to the feedstream quality available will determine the targeted operating conditions and product yields from LG2F processing. See Table 6.

TABLE 6
Targeting Middle Distillates for Diesel Blendstocks

| Middle Distillate Compound | Formula | TYPE | Boil Pt ° C. | Freeze Pt ° C. | Example Temp Ranges | | |
|---|---|---|---|---|---|---|---|
| N-PROPYLCYCLOPENTANE | C8H16 | N | 131.0 | −117.3 | X | | |
| 2,3,5-TR1METHYLHEXANE | C9H20 | P | 131.3 | −127.8 | X | | |
| ETHYLCYCLOHEXANE | C8H16 | N | 131.8 | −111.3 | X | | |
| 2,2-DIMETHYLHEPTANE | C9H20 | P | 132.7 | −113.0 | X | | |
| 2,2,3,4-TETRAMETHYLPENTANE | C9H20 | P | 133.0 | −121.1 | X | | |
| 2,2,3-TRIMETHYLHEXANE | C9H20 | P | 133.6 | −17.8 | X | | |
| 2,2-DIMETHYL-3-ETHYLPENTANE | C9H20 | P | 133.8 | −99.5 | X | | |
| 2,6-DIMETHYLHEPTANE | C9H20 | P | 135.2 | −102.9 | X | X | |
| 2,4-DIMETHYL-3-ETHYLPENTANE | C9H20 | P | 136.7 | −122.4 | X | X | |
| 2,2,5,5-TETRAMETHYLHEXANE | C10H22 | P | 137.5 | −12.6 | X | X | |
| 2,3,3-TRIMETHYLHEXANE | C9H20 | P | 137.7 | −116.8 | X | X | |
| 1,1-DIMETHYL-2-ETHYL-CYCLOPENTANE | C9H18 | N | 138.0 | −17.8 | X | X | |
| 2,2,3,3-TETRAMETHYLPENTANE | C9H20 | P | 140.3 | −9.9 | X | X | |
| 3,3,4-TRIMETHYLHEXANE | C9H20 | P | 140.5 | −101.2 | X | X | |
| 2,3,3,4-TETRAMETHYLPENTANE | C9H20 | P | 141.6 | −102.1 | X | X | |
| 4-METHYLOCTANE | C9H20 | P | 142.4 | −113.2 | X | X | |
| 3-ETHYLHEPTANE | C9H20 | P | 143.0 | −17.8 | X | X | |
| 2-METHYLOCTANE | C9H20 | P | 143.3 | −80.4 | X | X | |
| 3-METHYLOCTANE | C9H20 | P | 144.2 | −107.6 | X | X | X |
| 1-METHYL-1-n-PROPYL-CYCLOPENTANE | C9H18 | N | 146.0 | −17.8 | X | X | X |
| 3,3-DIETHYLPENTANE | C9H20 | P | 146.2 | −33.1 | X | X | X |
| ISOBUTYLCYCLOPENTANE | C9H18 | N | 148.0 | −115.2 | X | X | X |
| 1,1-DIETHYLCYCLOPENTANE | C9H18 | N | 150.5 | −17.8 | X | X | X |
| N-NONANE | C9H20 | PN | 150.8 | −53.5 | X | X | X |
| CYCLOOCTANE | C8H16 | N | 151.1 | 14.8 | X | X | X |
| CIS-1,2-DIETHYLCYCLOPENTANE | C9H18 | N | 153.6 | −118.0 | X | X | X |
| ISOPROPYLCYCLOHEXANE | C9H18 | N | 154.8 | −89.4 | X | X | X |
| 3,3,5-TRIMETHYLHEPTANE | C10H22 | P | 155.7 | −17.8 | X | X | X |
| N-BUTYLCYCLOPENTANE | C9H18 | N | 156.6 | −108.0 | X | X | X |
| N-PROPYLCYCLOHEXANE | C9H18 | N | 156.7 | −94.9 | X | X | X |
| 2,4-DIMETHYL-3-ISOPROPYL-PENTANE | C10H22 | P | 157.0 | −81.7 | X | X | X |
| 2,7-DIMETHYLOCTANE | C10H22 | P | 159.9 | −54.0 | X | X | X |
| 2,2,3,3-TETRAMETHYLHEXANE | C10H22 | P | 160.3 | −54.0 | X | X | X |
| 3,3,4-TRIMETHYLHEPTANE | C10H22 | P | 161.9 | −17.8 | X | X | X |
| ETHYLCYCLOHEPTANE | C9H18 | N | 163.3 | −17.8 | X | X | X |
| 5-METHYLNONANE | C10H22 | P | 165.1 | −87.7 | X | X | X |
| 4-METHYLNONANE | C10H22 | P | 165.7 | −98.7 | X | X | X |
| 2-METHYLNONANE | C10H22 | P | 167.0 | −74.7 | X | X | X |
| 3-METHYLNONANE | C10H22 | P | 167.8 | −84.8 | X | X | X |
| 1-METHYL-4-ISOPROPYL-CYCLOHEXANE | C10H20 | N | 170.7 | −17.8 | X | X | X |
| ISOBUTYLCYCLOHEXANE | C10H20 | N | 171.3 | −17.8 | X | X | X |
| TERT-BUTYLCYCLOHEXANE | C10H20 | N | 171.6 | −41.2 | X | X | X |
| N-DECANE | C10H22 | PN | 174.2 | −29.6 | X | X | X |
| CYCLONONANE | C9H18 | N | 178.4 | 11.0 | X | X | X |
| SEC-BUTYLCYCLOHEXANE | C10H20 | N | 179.3 | −17.8 | X | X | X |
| N-PENTYLCYCLOPENTANE | C10H20 | N | 180.5 | −83.0 | X | X | X |
| N-BUTYLCYCLOHEXANE | C10H20 | N | 180.9 | −74.7 | X | X | X |
| TRANS-DECAHYDRONAPHTMALENE | C10H18 | N | 187.3 | −30.4 | X | X | X |
| CIS-DECAHYDRONAPHTHALENE | C10H18 | N | 195.8 | −43.0 | X | X | X |
| N-UNDECANE | C11H24 | PN | 195.9 | −25.6 | X | X | X |
| N-HEXYLCYCLOPENTANE | C11H22 | N | 202.9 | −73.0 | X | X | X |
| N-PENTYLCYCLOHEXANE | C11H22 | N | 203.7 | −57.5 | X | X | X |
| N-DODECANE | C12H26 | PN | 216.3 | −9.6 | X | X | X |
| N-HEPTYLCYCLOPENTANE | C12H24 | N | 223.9 | −53.0 | X | X | X |
| N-HEXYLCYCLOHEXANE | C12H24 | N | 224.7 | −43.0 | X | X | X |
| 9-ETHYL-[TRANS-DECAHYDRO- | C12H22 | N | 225.0 | −17.8 | X | X | X |
| NAPHTHALENE] | | | | | | | |
| 9-ETHYL[CIS- | C12H22 | N | 232.8 | −17.8 | X | X | X |
| DECAHYDRONAPHTHALENE] | | | | | | | |
| 1-METHYL[TRANS-DECAHYDRO- | C11H20 | N | 235.0 | −17.8 | X | X | X |
| NAPHTHALENE] | | | | | | | |
| N-TRIDECANE | C13H28 | PN | 235.5 | −5.4 | X | X | X |
| BICYCLOHEXYL | C12H22 | N | 239.0 | 3.6 | X | X | X |
| 1-METHYL-[CIS-DECAHYDRO- | C11H20 | N | 243.0 | −17.8 | X | X | X |
| NAPHTHALENE] | | | | | | | |
| N-OCTYLCYCLOPENTANE | C13H26 | N | 243.5 | −44.0 | X | X | X |
| N-HEPTYLCYCLOHEXANE | C13H26 | N | 244.9 | −30.5 | X | X | X |
| N-TETRADECANE | C14H30 | PN | 253.6 | 5.9 | X | X | X |
| 1-ETHYL-[TRANS-DECAHYDRO- | C12H22 | N | 255.0 | −17.8 | X | X | X |
| NAPHTHALENE] | | | | | | | |
| 1-ETHYL[CIS-DECAHYDRO- | C12H22 | N | 260.0 | −17.8 | X | X | X |
| NAPHTHALENE] | | | | | | | |
| N-NONYLCYCLOPENTANE | C14H28 | N | 262.0 | −29.0 | X | X | X |
| N-OCTYLCYCLOHEXANE | C14H28 | N | 263.6 | −19.7 | X | X | X |
| N-PENTADECANE | C15H32 | PN | 270.7 | 9.9 | X | X | X |
| N-DECYLCYCLOPENTANE | C15H30 | N | 279.4 | −22.1 | X | X | X |
| N-NONYLCYCLOHEXANE | C15H30 | N | 281.5 | −10.2 | X | X | X |
| N-HEXADECANE | C16H34 | PN | 286.9 | 18.2 | X | X | X |
| N-UNDECYLCYCLOPENTANE | C16H32 | N | 295.8 | −10.0 | X | X | |
| N-DECYLCYCLOHEXAME | C16H32 | N | 297.6 | −1.7 | X | X | |
| N-HEPTADECANE | C17H36 | PN | 302.0 | 22.0 | X | | |
| N-OODECYLCYCLOPENTANE | C17H34 | N | 311.2 | −5.0 | X | | |
| N-UNDECYLCYCLOHEXANE | C17H34 | N | 313.2 | 5.8 | X | | |
| N-OCTADECANE | C18H38 | PN | 316.3 | 28.2 | X | | |
| N-TRrDECYLCYCLOPENTANE | C18H36 | N | 325.9 | 5.0 | X | | |
| N-DODECYLCYCLOHEXANE | C18H36 | N | 327.9 | 12.5 | X | | |
| N-NONADECANE | C19H40 | PN | 335.6 | 31.9 | X | | |
| N-TETRADECYLCYCLOPENTANE | C19H38 | N | 340.0 | 9.0 | X | | |
| N-TRIDECYLCYCLOHEXANE | C19H38 | N | 341.9 | 18.5 | X | | |
| N-EICOSANE | C20H42 | PN | 343.8 | 36.4 | X | | |
| N-PENTADECYLCYCLOPENTANE | C20H40 | N | 353.0 | 17.0 | X | | |
| N-TETRADECYLCYCLOHEXANE | C20H40 | N | 355.0 | 24.0 | X | | |

TABLE 7
Hydrocarbons to C9-14 Paraffins

| Carbon | Broad | Low Temp | Custom | High |
|---|---|---|---|---|
| # | Spectrum | Flowability | Blend | Cetane |
| 9 | X | X | | |
| 10 | X | X | X | |
| 11 | X | X | X | |
| 12 | X | X | X | X |
| 13 | X | X | X | X |
| 14 | X | X | X | X |
| 15 | X | | X | X |
| 16 | X | | X | X |
| 17 | X | | X | |
| 18 | X | | X | |
| 19 | X | | X | |
| 20 | X | | X | |
| 21 | X | | | |
| 22 | X | | | |
| 23 | X | | | |

In one embodiment, the LG2F Process is tailored to produce a narrow range of C9 to C14, high-cetane paraffins with few low-melting compounds, thereby minimizing any need for dewaxing. See Table 7. This product is a desirable diesel fuel blendstock due to its speed of starting, clean combustion and low temperature flowability.

### Aromatics

### EXAMPLES

### Diesel Blendstocks

This same fully-recycled LG2F Process can be operated at conditions to produce any targeted range (e.g. C₉₊) of hydrocarbons for use as middle distillate for diesel fuel blendstocks. The thermal olefination reaction creates a spectrum of C₂ to C₅ olefin-rich hydrocarbons, and the acid-catalyzed chemical reactor uses operating conditions which favor the C₉ to C₂₄₊ range of hydrocarbon compounds used in diesel fuel blendstocks. Selecting any segment of the C₂ to C₁₁₊ range of molecules output from the R2 catalytic reaction for recycle or aromatic reuse, and setting the appropriate operating conditions (T, P, WHSV) allows a tailored outcome of middle distillate with high cetane and low pour point values ideal for diesel fuel blendstocks. Byproducts of the reaction include methane, hydrogen and aromatic surplus. See Table 8a which depicts processing C5 (pentane) to diesel, and Table 8b which depicts processing C2 (ethane) to diesel using the LG2F R1→R2→recycle process.

TABLE 8a
LG2F Production of Diesel Blendstock from C5 (pentane) feedstock

| | Process Step | | | | | | | |
|---|---|---|---|---|---|---|---|---|
| | 1 | 2 | 3 | 4 | 5 | 6 | 7a | 7b |
| | | | Reactor 2 | | | | | |
| G2L | | Reactor 1 | Out, | | | | | |
| Recycle Mass | | Out, 76% | 100% | Flash | Recycle | Lights | | Diesel |
| Balance (lbs/hr) | Feed | Conv | Conv | Tops | Loop | Purge | Aromatics | Blendstock |
| H2 | | 0.18 | 0.18 | 0.18 | | 0.18 | | |
| C1 | | 19.04 | 19.04 | 19.04 | | 19.04 | | |
| C2 | | 50.11 | 16.52 | 16.52 | 16.52 | | | |
| C3 | | 31.26 | 2.2 | 2.2 | 2.2 | | | |
| C4 | | 9.41 | 1.11 | 1.11 | 1.11 | | | |
| C5 | 100 | 42.3 | 39.17 | 39.17 | 39.17 | | | |
| C6 | | | | | | | | |
| C7 | | | | | | | | |
| C8 | | | | | | | | |
| C9 | | | | | | | | |
| C10 | | | | | | | | |
| C11 | | | 0.02 | | | | | 0.02 |
| C12+ | | | 65.48 | | | | | 65.48 |
| A6 | | 3.86 | 4.58 | | | | 4.58 | |
| A7 | | 1.46 | 3.09 | | | | 3.09 | |
| A8 | | | 3.03 | | | | 3.03 | |
| A9 | | | 1.42 | | | | 1.42 | |
| A10 | | | 1.08 | | | | 1.08 | |
| A11 | | | 0.72 | | | | 0.72 | |
| Unknown | | 1.36 | 1.36 | | | | | 1.36 |
| Total | 100 | 158.98 | 159 | 78.22 | 59 | 19.22 | 13.92 | 66.86 |

TABLE 8b
LG2F Production of Diesel Blendstock from C2 (ethane) feedstock

| | Process Step | | | | | | | |
|---|---|---|---|---|---|---|---|---|
| | 1 | 2 | 3 | 4 | 5 | 6 | 7 | 8 |
| LG2F using | | | | | | | | |
| C2 w/Recycle | | | | | | Lights | Light | |
| (lb/hr) | Feed | R1 Out | R2 Out | Flash Tops | Recycle | Purge | Aromatics | Diesel |
| H2 | | 1.60 | 1.60 | 1.60 | | 1.60 | | |
| C1 | | 14.34 | 14.67 | 14.67 | | 14.67 | | |
| C2 | 100 | 114.26 | 115.20 | 115.20 | 115.20 | | | |
| C2= | | 85.27 | 2.44 | 2.44 | 2.44 | | | |
| C3 | | 0.24 | 0.24 | 0.24 | 0.24 | | | |
| C3= | | 1.77 | | | | | | |
| C4 | | 0.41 | 1.24 | 1.24 | 1.24 | | | |
| C4= | | 0.26 | 0.12 | 0.12 | 0.12 | | | |
| C5 | | | 0.16 | 0.16 | 0.16 | | | |
| C6 | | | | | | | | |
| C7 | | | | | | | | |
| C8 | | | | | | | | |
| C9 | | | | | | | | |
| C10 | | | | | | | | |
| C11 | | | 0.02 | | | | | 0.02 |
| C12+ | | | 72.07 | | | | | 72.07 |
| A6 | | 0.37 | 1.16 | | | | 1.16 | |
| A7 | | | 2.71 | | | | 2.71 | |
| A8 | | | 3.33 | | | | 3.33 | |
| A9 | | | 1.57 | | | | | 1.57 |
| A10 | | | 1.19 | | | | | 1.19 |
| A11 | | | 0.79 | | | | | 0.79 |
| Unknown | | 0.89 | 0.89 | | | | | 0.89 |
| Total | 100 | 219.41 | 219.41 | 135.68 | 119.41 | 16.27 | 7.20 | 76.53 |

Note:
“=” used to represent olefins; “C2 =” is ethylene

LG2F Process Flexibility

The LG2F process conditions are easily convertible to switch processing methods which offers a unique capability to adjust the production of key transportation fuels depending upon ever-changing market conditions. A particular feature of the LG2F process is the option to produce gasoline blendstocks at one set of operating conditions and/or switch to produce middle distillate blendstocks at a different set of (R2) chemical reactor operating conditions. Depending upon the availability of downstream processing often available at refining plants, the timing of the process switching can be tailored using distinctive cuts to eliminate the need for any distillation of the blendstocks. In one embodiment, the process could be solely devised to produce middle distillate grade product blendstocks of a high cetane and net heat value. In a different embodiment, the process could be solely devised to produce higher octane gasoline blendstocks. In yet another embodiment, the process could be set to produce higher octane gasoline blendstocks during one period, then switched and reconfigured to produce middle distillate blendstocks in another period. In yet another embodiment, the process could be set to produce a full spectrum of for example, C₅₊ or C₆₊ or C₇₊ fuel products which could be distilled downstream for different commercial uses. Once again, the preferred end product of the reaction (e.g. the targeted performance requirements of a fuel blendstock) may have a determining factor on the ideal operating conditions (T,P,WHSV) and choice of the R2 catalyst.

The LG2F process also offers a wide range of modular configurations (e.g. to eliminate benzene or increase octane or increase energy density or increase net heat of combustion or lower vapor pressure) when processing C2 to C5 light gases which allows for the tailoring of the operating conditions resulting in a specified composition of gasoline blendstock. In one embodiment, the LG2F R1+R2 reaction with recycling is specified to produce only C7 to C10 aliphatic and aromatic hydrocarbons between the boiling point range of 85° (above benzene) up to 200° C. This results in a well-balanced high-octane gasoline blendstock with no benzene. In another embodiment, the LG2F R1+R2 reaction with recycling is specified to produce C5 to C10 aliphatic (favoring paraffins and olefins) with virtually no aromatics. This results in a lower octane blendstock, but with higher volumetric yields. In another embodiment, the LG2F R1+R2 reaction is specified to produce primarily C7 to C10 high octane aromatics with only a minor content of aliphatic hydrocarbons. This results in a high-octane gasoline blendstock, in the absence of benzene, and a high energy density.

This modular functionality in designing tailored hydrocarbon product streams from C2-C5 light gas streams is a major feature of this invention. This tailoring can be applied to adjust to ever-changing market conditions and locational arbitrage opportunities. The LG2F R1 and R2 reactors can operate independently or in an integrated fashion. Any available source of olefins can be used in the R2 reaction once the feedstock composition is assessed for the ideal temperature, pressure and reaction time for a given product specification. The product high (final) boiling point is specified by the R2 operating conditions and the product low (initial) boiling point is set by the flash drum cut point which eliminates any need distillation.

### Combined I2FE and LG2F

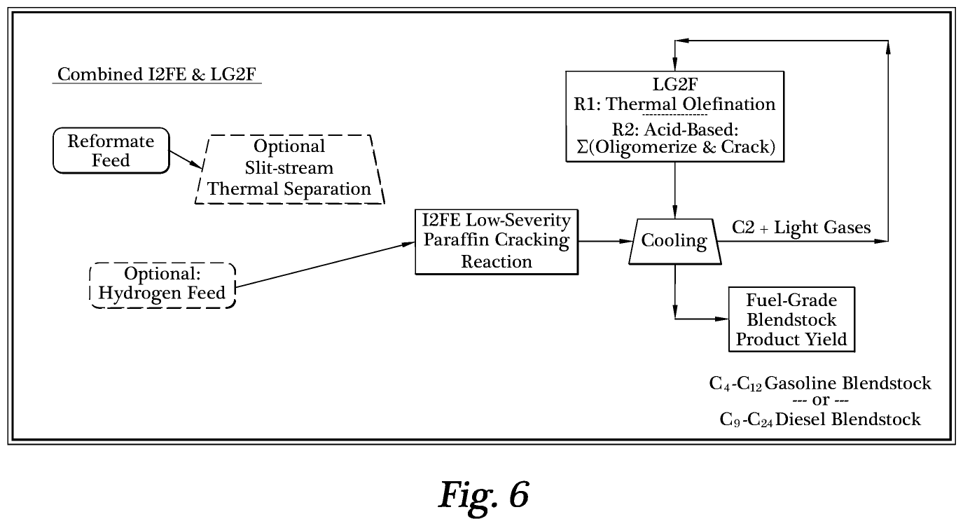

Another aspect of the LG2F Process is the ability to combine the process with another process which provides a source of C2-5 hydrocarbons useful as a feed to the LG2F Process. This other process is described in a co-pending application, U.S. Ser. No. 16/242,465, also owned by Applicant. This other process is referred to as the process for Increase to Fuel Economy, or “I2FE”. This combined process is presented in FIG. 6.

The I2FE process can be designed to consume a small amount of hydrogen to maintain the longevity of the metal catalyst. Depending upon design configurations, hydrogen byproduct from LG2F may offset on-purpose hydrogen consumed in I2FE, if these two processes are used together. The design of both units can be balanced and optimized to be hydrogen natural or a net producer of hydrogen, depending upon the needs of the business operation. See FIG. 6.

In this combined embodiment, the LG2F Process converts the clean light gas compounds (C₂₊) specifically from the I2FE process to produce C6+ blendstocks using thermal olefination (R1) followed by a multi-iterative acid-catalyzed oligomerization, cyclization and cracking reaction (R2) in a single or multi-bed reactor configuration with a recycle loop. In one embodiment, for example, the Process is used to yield any range of C₉ to C₂₄₊, zero-sulfur, middle distillate compounds with effective performance properties for use in diesel fuel and other transportation fuel blendstocks. The same process can be performed targeting a narrower range of middle distillate compounds such as C10-C20, or C12-C18, or C9-C14, etc. depending upon the performance requirements of the finished product. A byproduct of this process depending upon the configuration is unused hydrogen, methane and surplus aromatics.

Another embodiment of this LG2F invention converts the clean light gas compounds (C₂₊) specifically from the I2FE process, with or without reformer off-gases, to produce gasoline range blendstocks using only thermal olefination and a multi-iterative acid-catalyzed oligomerization, cyclization and cracking reaction in a single or multi-bed reactor configuration with a recycle loop. This process is designed to handle excess hydrogen to yield any C₄ to C₁₂ range gasoline compounds (i.e., paraffins, olefins and aromatics) for use with other gasoline blendstocks. All gasoline products in this embodiment are very-low benzene, sulfur-free and nitrogen-free. A byproduct of this process depending upon the configuration is unused hydrogen, methane and surplus aromatics.

### I2FE to C6-11 Gasoline Blendstock

Another embodiment of this LG2F invention converts the clean light gas compounds (C₂₊) specifically from the I2FE process to produce gasoline range blendstocks using thermal cracking (R1) and a multi-iterative acid catalyzed reaction (R2) in a single or multi-bed reactor configuration along with a recycle loop. This process is designed without excess hydrogen to yield C₄ to C₁₂ range gasoline compounds (i.e. paraffins, olefins and aromatics) for use with other gasoline blendstocks. All gasoline products in this embodiment are sulfur-free and nitrogen-free. Alternatively, this process is designed to provide excess hydrogen for reuse. Depending upon the configuration methane and surplus aromatics may be byproducts of the reaction.

Production of Gasoline/Diesel Fuels from CH₄

The LG2F Process can be tailored to utilize a specialized high-temperature thermal cracking process, referred to herein as methane thermal olefination (“MTO”) with light gas feedstocks comprised primarily of CH₄. C₂H₄ is the key product of MTO and may have viable uses as a valuable feedstock in the petrochemical marketplace (primarily to make polymers) bypassing the need for traditional ethane/propane cracking methods.

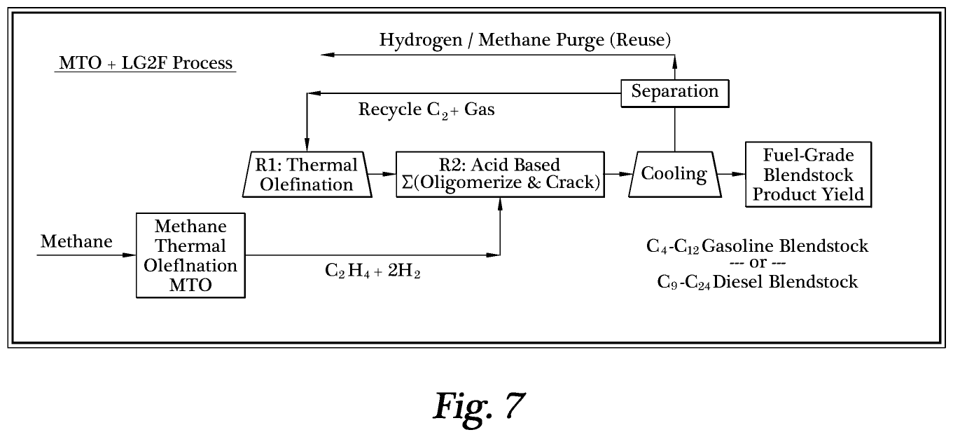

The LG2F Process for performance of MTO is shown in FIG. 7. The MTO cracking unit is operated above 900° C. at pressures above 150 psig without the requirements for a catalyst to produce high concentrations (e.g. >80%) of C₂H₄. The olefin-rich C₂H₄ produced is processed first by the LG2F catalytic reaction (R2) with an appropriate recycling loop back to C2+ thermal olefination to produce any targeted range of C₄-C₁₂ gasoline blendstocks or C₉-C₂₄ diesel blendstocks. Methane and hydrogen are unreacted in the LG2F process and purged for reuse. See FIG. 7.

The basic chemistry of MTO is 2 CH₄→C₂H₄+2 H₂. For optimal selectivity and conversion of olefins, methane recycling back through MTO may be utilized. C₂H₄ is a prime olefin produced by MTO which provides the ideal feedstock to the LG2F process by using the C₂₊ catalytic reactor (R2) as illustrated herein. Hydrogen does not react in the LG2F process and is extracted for reuse. This process operates without any requirement for O₂ or steam or water gas shift reactions. This process does not require complex multi-stage distillations or cryogenic separation or selective hydrogenation. This process produces fuel grade gasoline and diesel products without ultra-fine purity typical of petrochemical processes. Alkane byproducts from MTO do not react in the R2 catalytic reaction but will be recycled through the R1 thermal olefination process to maximize yield of fuel grade blendstocks. Some variations of the MTO process may utilize specialized catalysts including those with compounds to promote or accelerate the reaction.

### Alkene Feedstocks

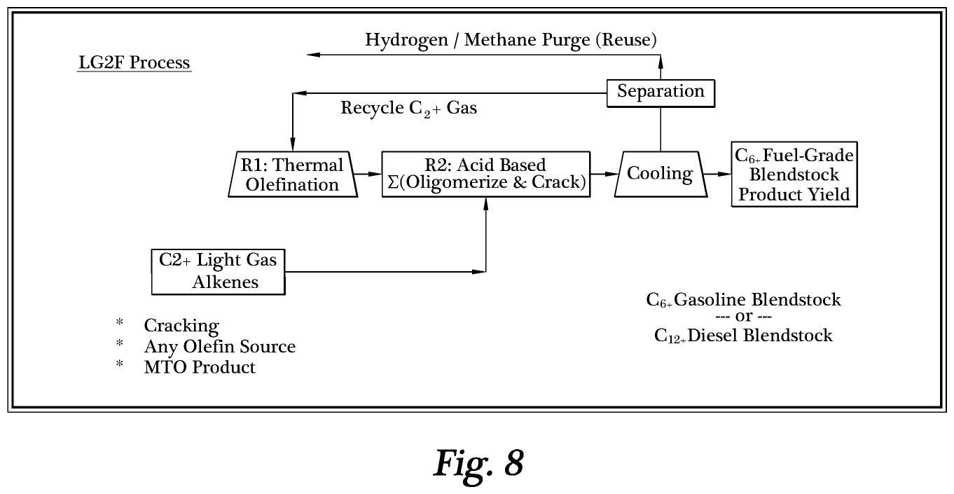

The LG2F Process is also useful with light alkene gases, including ethylene, propylene, butylene and iso-butylene. Typical Cat-Cracked Gasoline byproducts include C₃ alkenes and LPG which are desirable feedstocks to the LG2F process for upgrading to higher value gasoline or diesel fuel blendstocks. The presence of alkenes in the feedstock may favor the use first of the acid-catalyzed chemical reaction, as exemplified in FIG. 8.

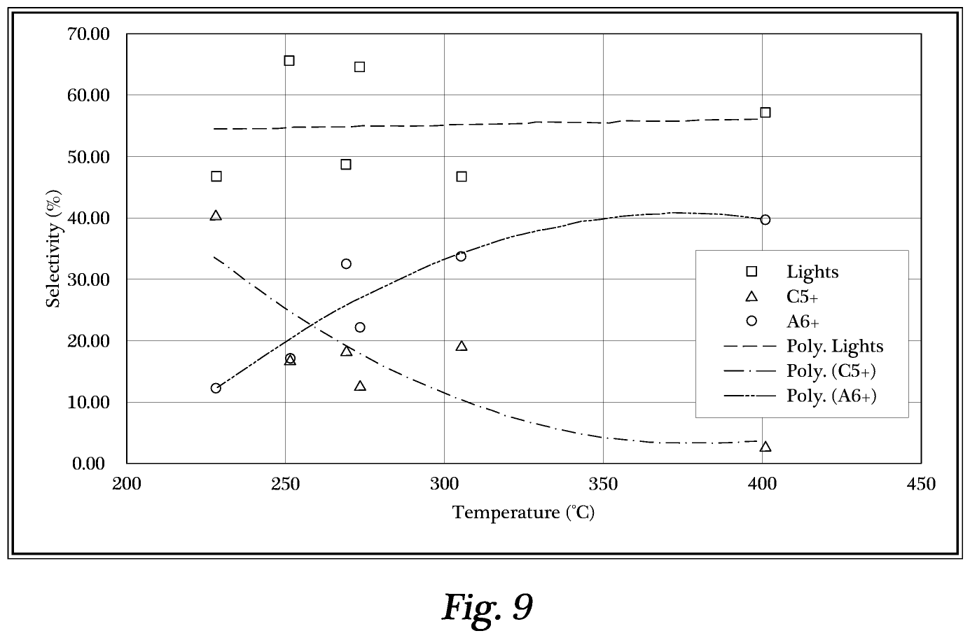

As an illustration of the processing of alkene gases, a single pass yield of the C₂+ acid-based chemical reaction, shown in FIG. 9, is from a C₃ olefin feedstock and demonstrates the production of gasoline grade compounds. This reaction was made at 45 psig and 3 WHSV across a range of temperatures. As illustrated, the aromatic hydrocarbon content (A₆₊) varied by the reaction temperature which can be used to increase octane values of gasoline blendstocks.

Another embodiment of this LG2F invention, receives the byproduct light gases from the catalytic cracking process (often containing C₃₊ olefinic compounds, e.g. propylene) to produce gasoline range blendstocks. In this embodiment, the olefinic feed bypasses the thermal olefination reactor and goes straight into the multi-iterative acid-catalyzed reaction in a single or multi-bed reactor configuration with a recycle loop. This process is designed to provide excess hydrogen to yield C₆ to C₁₁ range gasoline compounds (i.e. paraffins, olefins and aromatics) for use with other gasoline blendstocks. All gasoline products in this embodiment are very-low benzene, sulfur-free and nitrogen-free. A byproduct of this process is unused hydrogen.

Another embodiment of this LG2F invention receives the byproduct light gases from the catalytic cracking process (often containing C₃₊ olefinic compounds, e.g. propylene) to produce diesel range fuel blendstocks. This embodiment bypasses the initial thermal olefination and goes straight into the multi-iterative acid-catalyzed reaction in a single or multi-bed reactor configuration with an R2 catalyst tailored for the feedstream before re-entering the LG2F recycle loop. This process is designed to provide excess hydrogen and to yield any specified range of C₄ to C₁₂ gasoline blendstocks or C₉ to C₂₄ middle distillate for use as diesel fuel blendstocks. A byproduct of this process is unused hydrogen.

The foregoing processes are examples of a range of processes using alkene feeds, further including the following:

C₂+ alkene gas streams exiting the catalytic cracking unit are transformed to C₆₊ gasoline constituents first via LG2F chemical reaction (R2), followed by a recycle loop that restarts thermal olefination and a chemical reaction loop resulting in higher liquid gasoline yields;

C₂+ light hydrocarbon streams with primarily olefinic compounds are merged to increase the available volume of light gas compounds for conversion via R2 processing with recycle loops to R1+R2 to produce gasoline blendstocks using the LG2F process;

C₂+ light hydrocarbon streams with primarily olefinic compounds are merged to increase the available volume of light gas compounds for conversion to light gas oil or diesel fuel blendstocks using LG2F.

### Reducing Benzene

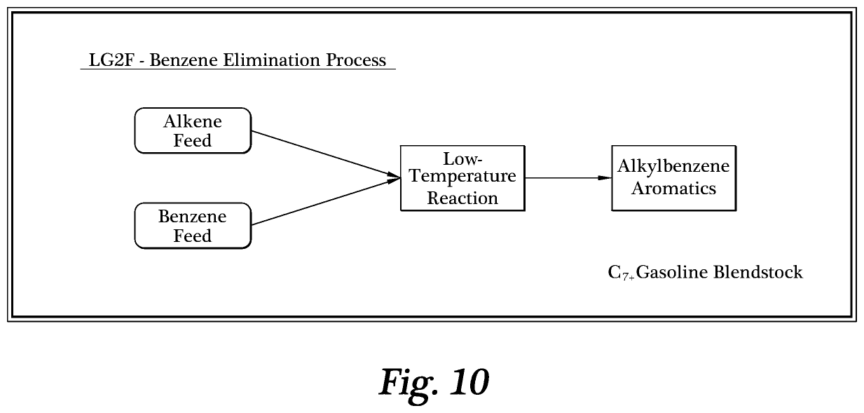

Another major feature of this light gas transformation to transportation fuel is the selective reduction of benzene, which makes the resulting products excellent for gasoline blending due to low specification limits placed on benzene for use in fuels. In the case where there is an unwanted surplus of benzene-rich C6+ aromatics extracted by liquid-vapor knockout from the R1 thermal olefination effluent, an added feature of LG2F is to combine light alkene compounds (e.g. C2-C3) from the R1 reaction with the surplus C6+ aromatic compounds into a simple low-temperature acid-catalyzed reaction to create alkyl-benzenes. This processing will convert benzene via electrophilic substitution to become productive gasoline grade blendstocks that adhere to existing limitations in gasoline specifications for high-octane aromatic compounds. This process may utilize aluminum chloride and hydrogen chloride catalysts. This process will further increase the value of the gasoline blendstock. See FIG. 10.

### Dewaxing

Another aspect of this invention is a simplified method to dewax paraffinic compounds from C₁₄ to C₄₀ hydrocarbon streams using a single-stage, low-severity, acid-catalyzed reaction process to both hydrocrack and hydrotreat middle-to-heavy grade distillate feedstocks to produce a higher-value, higher-grade middle distillate with higher fuel performance properties.

Catalytic dewaxing is typically referred to as a process that selectively removes C₁₄₊ paraffinic compounds from middle- to heavy-distillate hydrocarbon streams. This technology is usually applied to hydrocarbons used in diesel fuel and heating oils to improve its physical properties including cloud point, pour point and cold flowability. Increasing quality reduces the need of using fuel additives to improve properties and allows for more detailed control of blending specifications. The primary alternative technology to catalytic dewaxing is solvent based dewaxing which applies a solvent extraction method to heavy paraffinic compounds that preserves the chemical structure.

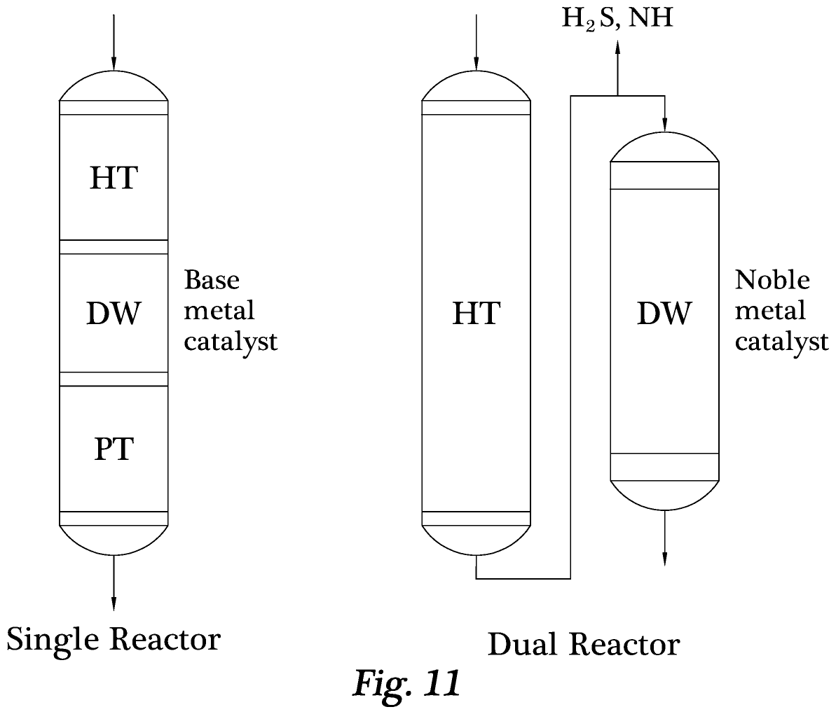

Configurations of traditional dewaxing units vary but are most often categorized in two categories: a single or dual bed reactor. The choice in configuration depends on preference for hydrotreating integration into the dewaxing catalytic system. The inlet streams have higher concentrations of sulfur and nitrogen which will deactivate noble metal catalysts. So, a hydrotreating bed is typically integrated before the dewaxing catalyst to ensure minimal degradation of performance. See, FIG. 11.

### Traditional Dewaxing Methods

Traditional refinery dewaxing catalysts are nickel- or platinum-based selective zeolites, which is a molecular sieve catalyst. By controlling pore size, these methods control the types of molecules that enter the catalytically active sites. Specifically, the pore sizes are set to allow n-paraffinic compounds but not isoparaffinic compounds (0.6 nm). Traditional hydrotreating catalysts commonly use Ni/Mo metal combination to perform the hydrogenation of nitrogen and sulfur-based compounds. The configuration of these catalyst depends on the level of protection needed in a dewaxing unit. If there are lower than normal catalyst poisons, then a single reactor can be used with a protective bed above the dewaxing bed. However, if poisons are an issue then a separate hydrotreating bed will be beneficial to sustained catalyst life. A comparison between typical single and dual bed catalysts are show in Table 9.

TABLE 9

| Product distribution | Single stage | Second stage |
|---|---|---|
| (wt %) | (SDD-800) | (SDD-821) |
| C1-C4 | 4.3 | 0.2 |
| C5-177° C. | 9.2 | 5.9 |
| 177° C.+ | 86.7 | 94.5 |
| Total | 100.2 | 100.6 |

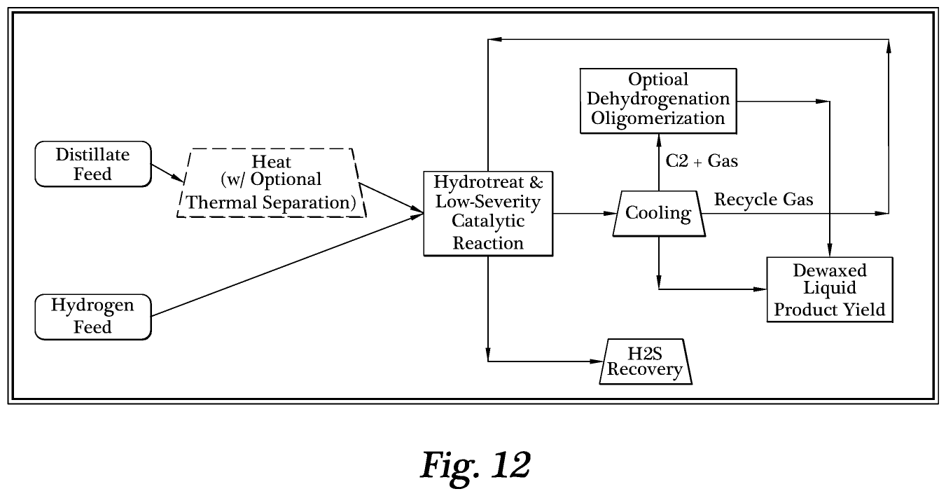

Traditional methods for dewaxing require a separation between two catalytic beds with one performing hydrotreating and the other selectively cracking n-paraffinic compounds. Noble metal catalysts propose too high of a risk for poisoning from hydrogen sulfide and ammonia, hence the removal of these gases before dewaxing. However, base metal catalysts lack the activity needed to dewax a hydrocarbon stream effectively and require larger utility costs. See FIG. 12.

This invention utilizes a unique, low-severity method for hydrocracking the C₁₄₋ to C₄₀₊ paraffins or any targeted range of n-paraffins compounds using a specialized zeolite catalyst with the capability to simultaneously hydrotreat the feedstream thereby removing the sulfur and nitrogen-based compounds and cracking the low-melting paraffins in a single step process. This unique method reduces total costs of processing and eliminates the need for additives used in the field. The main target is cracking broad scope n-paraffinic compounds since even n-tetradecane (C₁₄) melts above low ambient temperatures. Having even a single branch significantly reduces the melting point by ˜80 F while still having a cetane value of 67.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected.

1. A process for converting methane and C₂₋₅ alkanes to a broad-range of fuel products constituting higher-value C₅₋₂₄₊ hydrocarbon fuels or fuel blendstocks, comprising:
passing methane through a methane thermal olefination reactor operating without a dehydrogenation catalyst and without steam, the methane thermal olefination reactor operating at a temperature of at least 900° C. and a pressure of at least 150 psig to produce an ethylene effluent stream comprising greater than 80 wt % ethylene;
passing a C₂₋₅ feedstream comprising at least 90 wt % C₂₋₅ feed alkanes through a C₂₋₅ thermal olefination reactor operating without a dehydrogenation catalyst and without steam, the C₂₋₅ olefination reactor operating at a temperature, pressure and space velocity to convert at least 80% of the C₂₋₅ feed alkanes to product olefins in a C₂₋₅ effluent stream;
passing the ethylene effluent stream and the C₂₋₅ effluent stream through an oligomerization reactor containing a zeolite catalyst and operating at a temperature, pressure and space velocity to crack, oligomerize and cyclize the olefins in the ethylene effluent stream and in the C₂₋₅ effluent stream to form an effluent oligomerization stream comprising the fuel products; and
recovering the fuel products from the effluent oligomerization stream.

2. The method of claim 1 in which the effluent oligomerization stream also comprises methane, the method further comprising passing the methane from the effluent oligomerization stream through the methane thermal olefination reactor.

3. A process for converting methane and C₂₋₅ alkanes to a broad-range of fuel products constituting higher-value C₅₋₂₄₊ hydrocarbon fuels or fuel blendstocks, comprising:
passing the methane through a methane thermal olefination reactor operating without a dehydrogenation catalyst and without steam, the methane thermal olefination reactor operating at a temperature of at least 900° C. and a pressure of at least 150 psig to produce an ethylene effluent stream comprising greater than 80 wt % ethylene;
passing a C₂₋₅ feedstream comprising at least 90 wt % C₂₋₅ feed alkanes through a C₂₋₅ thermal olefination reactor to produce a C₂₋₅ effluent stream comprising C₂₋₅ alkanes and C₂₋₅ olefins;
passing the ethylene effluent stream and the C₂₋₅ effluent stream through an oligomerization reactor, the oligomerization reactor containing a zeolite catalyst and operating at a temperature, pressure and space velocity to crack, oligomerize and cyclize the olefins in the ethylene effluent stream and the olefination effluent stream to form an effluent oligomerization stream comprising the fuel products and C₂₋₅ alkanes; and
separating C₂-4 alkanes from the effluent oligomerization stream and recycling the separated C₂-4 alkanes through the C₂₋₅ thermal olefination reactor,
the C₂₋₅ thermal olefination reactor operating at a temperature, pressure and space velocity to convert at least 80 wt % of the C₂₋₅ feed alkanes to olefins.

4. The method of claim 3 in which the effluent oligomerization stream also comprises methane, the method further comprising passing the methane from the effluent oligomerization stream through the methane thermal olefination reactor.

5. A process for converting methane to ethylene, comprising:
passing the methane through a methane thermal olefination reactor operating without a dehydrogenation catalyst and without steam, the methane thermal olefination reactor operating at a temperature of at least 900° C. and a pressure of at least 150 psig to produce an effluent comprising greater than 80 wt % ethylene.
